(19) United States
(12) Patent Application Publication
(10) Pub. No.: US 2019/0390235 A1
(43) Pub. Date: Dec. 26, 2019

(54) Processes for the Preparation of Dasotraline and Intermediates Thereof

(71) Applicant: Apotex Inc., Toronto (CA)

(72) Inventors: Fabio E. S. Souza, Mississauga (CA); Prabhudas Bodhuri, Torrance, CA (US); Eduardo Gustavo Cammisa, Markham (CA); Gamini Weeratunga, Ancaster (CA); Allan W. Rey, Brantford (CA)

(21) Appl. No.: 16/438,801

(22) Filed: Jun. 12, 2019

Related U.S. Application Data
(60) Provisional application No. 62/689,326, filed on Jun. 25, 2018.

(51) Int. Cl. C12P 13/00

(52) U.S. Cl. CPC C12P 13/001; C12Y 206/01018

(57) ABSTRACT

The present invention provides processes for the preparation of Dasotraline, as well as intermediates useful in the preparation thereof. In particular, processes are provided for the enzymatic transamination of a compound of Formula (2) to afford Dasotraline.

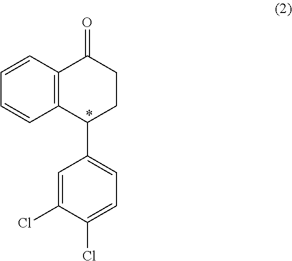 [SMILES: O=C1CCC(c2ccc(Cl)c(Cl)c2)c2ccccc21]

### CROSS-REFERENCE TO RELATED APPLICATION

This application claims the benefit of U.S. Provisional Patent Application No. 62/689,326, filed Jun. 25, 2019, the disclosure of which is hereby incorporated in its entirety by reference.

### TECHNICAL FIELD

The present invention relates to processes for the preparation of Dasotraline (1) and intermediates used in the preparation thereof.

### BACKGROUND

Dasotraline (1), or (1R,4S)-4-(3,4-dichlorophenyl)-1,2,3,4-tetrahydro-1-naphthalenamine, acts as a dual dopamine and norepinephrine reuptake inhibitor (DNRI), and is undergoing evaluation as a treatment for attention deficit hyperactivity disorder (ADHD) as its hydrochloride salt.

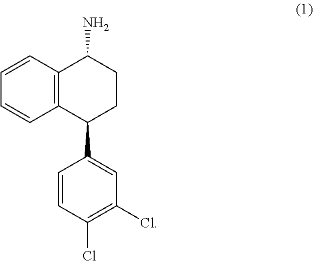 [SMILES: N[C@@H]1CC[C@@H](c2ccc(Cl)c(Cl)c2)c2ccccc21]

Scheme 1 depicts a known method of preparing the hydrochloride salt of Dasotraline (1) that is described in WO 2004/024669 A1 and Han, Z. et al. Org. Process Res. Dev. 2007, 11, 726. In this method, chiral (S)-tetralone (E) was prepared from racemic tetralone (A) by chromatographic separation of the sulfinyl imine diastereomers (C), followed by hydrolysis. The resulting (S)-tetralone was converted to a diastereomeric mixture of N-formyl amines (F), which were separated by chromatography to give the desired (1 R,4S)-trans-diastereomer (G), which afforded Dasotraline (1) as the hydrochloride salt following acidic hydrolysis. However, there are limitations associated with this process that impede its use on an industrial scale. These limitations include a low overall yield of <2% starting from racemic tetralone (A), which is due, in part, to the loss of at least 50% of the undesired stereoisomer in each of the two steps that generate chirality, as well as the hydrolytic sensitivity of intermediate sulfinyl imine (C). Also impeding the use of this process on an industrial scale is the necessity for two chromatographic separations to isolate diastereomeric intermediates (D) and (G) from mixtures (C) and (F), respectively, particularly owing to the reported sensitivity of sulfinyl imine (C) to hydrolysis.

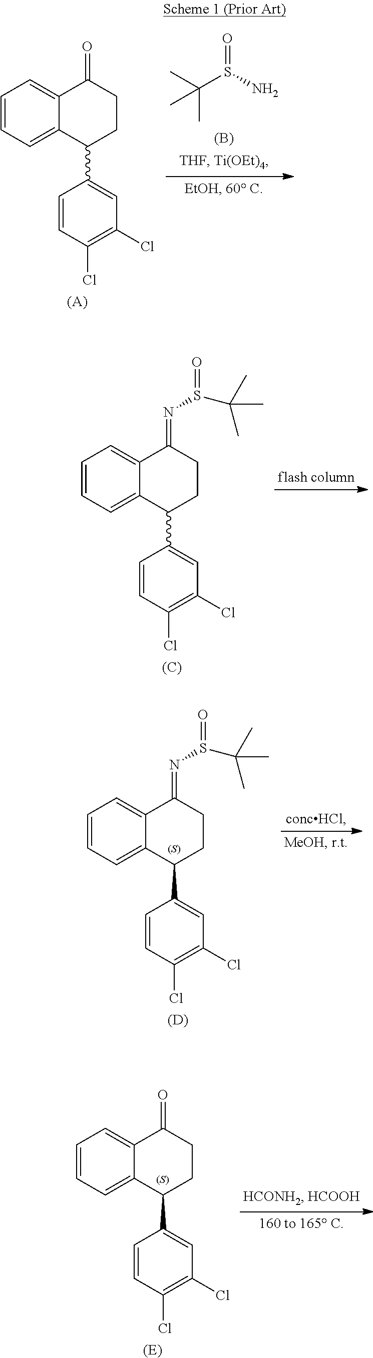

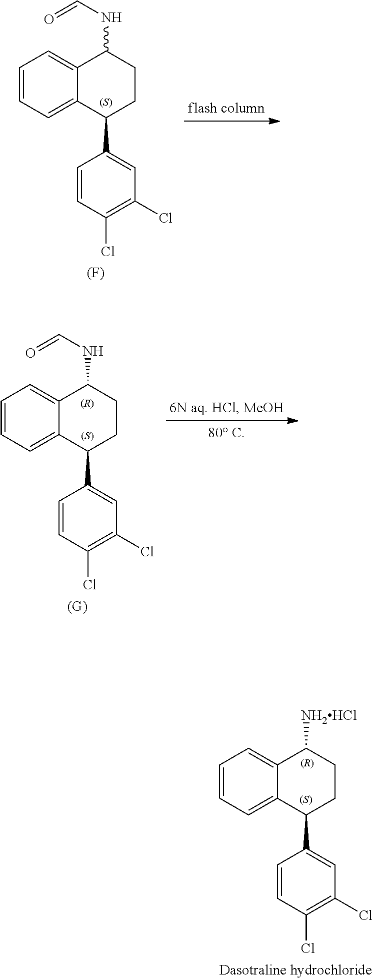

In Han, Z. et al. Org. Proc. Res. Dev. 2007, 11, 726, improvements to the route disclosed in WO 2004/024669 A1 are presented for the multi-kilogram production of Dasotraline hydrochloride. In this large scale synthesis, chiral sulfinylimine (D) is prepared from (S)-tetralone (E) and undergoes direct stereoselective reduction to the corresponding sulfonamide followed by hydrolysis to afford Dasotraline (1) in 50-56% yield from (E), without isolation of any sensitive intermediates. However, this approach suffers from the requirement for stoichiometric amounts of costly reagents such as sulfinamide (B) and 9-borabicyclo[3.3.1]nonane (9-BBN), which are necessary to achieve the highest levels of stereoselectivity.

Further syntheses of Dasotraline (1) are disclosed in Thalen, L. K. et al. Chem. Eur. J. 2009, 15, 3403 and WO 2007/115185 A2. Utilizing different substrates as starting materials, both of these approaches involve asymmetric catalytic hydrogenation of a precursor bearing one fixed chiral center. A key requirement of this approach is the use of costly homogenous catalyst systems such as the ‘Crabtree catalyst’ ([C₈H₁₂IrP(C₆H₁₁)₃C₅H₅N]PF₆) or Rh-Norphos, which can be difficult to remove due to their solubility in the reaction conditions. This is of particular relevance in the commercial production of Dasotraline (1), as residual transition metals must be controlled to very low levels in pharmaceutical products.

WO 2008/065177 A1 discloses processes for the preparation of desmethylsertraline, which corresponds with the (1 S,4S)-cis-isomer of Dasotraline. Scheme 2 depicts one approach from this disclosure that is used to produce desmethylsertraline involving reductive amination of racemic tetralone (A) with p-methoxybenzylamine, to selectively afford an equimolar mixture of the (1 S,4S)— and (1 R,4R)-cis-isomers of benzylated amine (J), followed by debenzylation and resolution of the resulting mixture of cis-amines using (+)-tartaric acid to yield desmethylsertraline as the (+)-tartrate salt. This approach to an isomer of Dasotraline suffers from many of the same impediments associated with the known methods of producing Dasotraline, including a low overall yield of <3% starting from racemic tetralone (A).

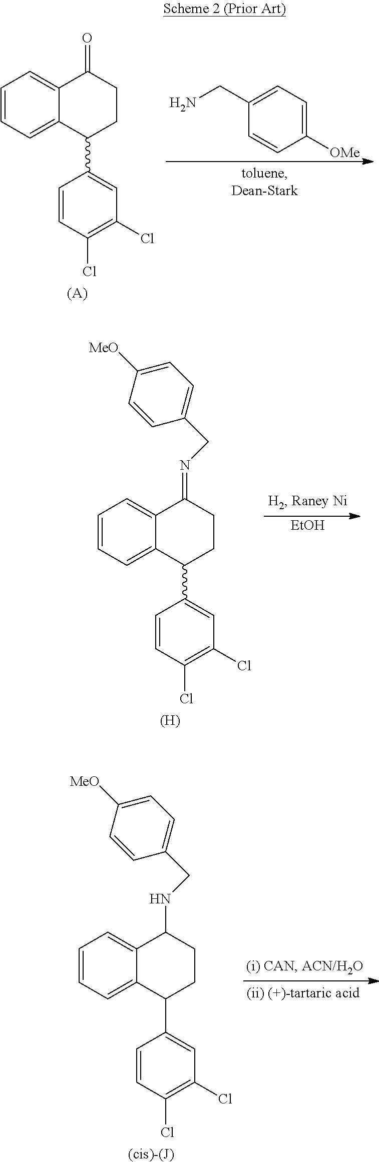

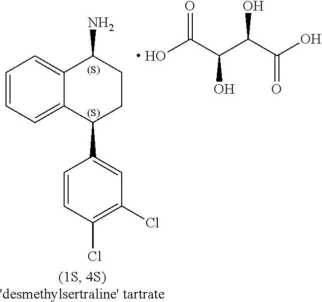

Accordingly, there remains a need in the art for improved processes for the preparation of Dasotraline (1), and the intermediates used in such preparations, that are more amenable to scale-up and use on a commercial scale.

### SUMMARY OF THE INVENTION

The present invention provides an improved process for the preparation of Dasotraline (1), or a salt thereof, from the compound of Formula (2) via an enzymatic transamination as depicted in Scheme 3.

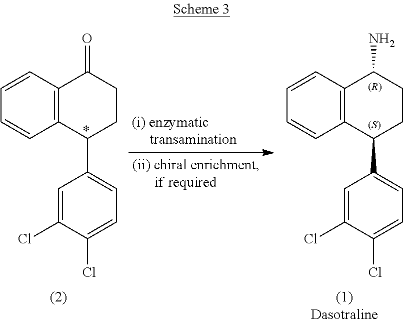

wherein
the carbon atom marked with “*” is racemic, substantially racemic, or enantiomerically enriched in the (S)-configuration.

As shown in Scheme 3, Dasotraline (1) may be prepared by (R)-selective enzymatic transamination of the compound of Formula (2). In this process, the enzymatic transamination is carried out using an (R)-selective ω-transaminase in the presence of an amine donor (a compound capable of providing an amino (NH₂) group to an amine acceptor in a transamination reaction mediated by an ω-transaminase enzyme) and a co-factor, preferably, a pyridoxal-5′-phosphate or another member of the vitamin B6 family and their phosphorylated counterparts.

In a preferred embodiment, the compound of Formula (2) has the required (S)-configuration at the C-4 position (*), allowing for direct access to Dasotraline (1) in a single step. Application of the process of the present invention using a chirally pure (S)-isomer of the compound of Formula (2) can provide Dasotraline (1) hydrochloride having high chiral purity (greater than 99% and preferably greater than 99.5%) in a single step in yields comparable to the multi-step process starting from the same chiral tetralone that is described in Org. Proc. Res. Dev. 2007, 11, 726, thereby providing a simplified and improved route to Dasotraline (1) that avoids the use of stoichiometric quantities of transition metal reagents and hydrolytically sensitive intermediates and reagents.

Alternatively, when a compound of Formula (2) having lower chiral purity in favor of the (S)-isomer, including racemic and substantially racemic material, is used, chiral enrichment procedures, can be employed following transamination to provide Dasotraline (1).

As shown in Scheme 4, in preferred embodiments of the processes of the present invention, the compound of Formula (2) may be prepared by oxidation of the amine of Formula (4), or a salt thereof, followed by hydrolysis of the resulting imine of Formula (3).

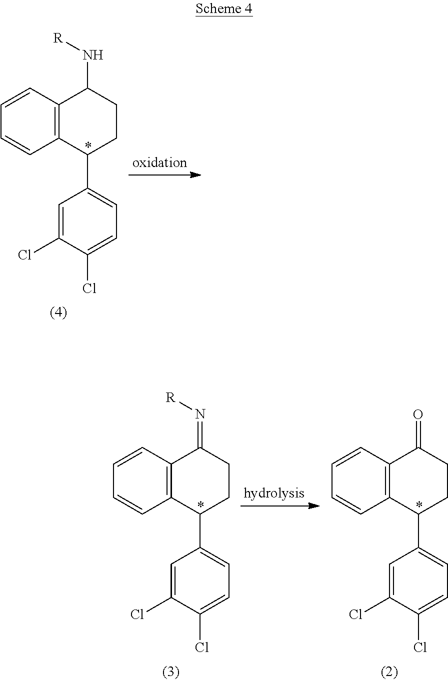

wherein

R is a C₁-C₆ alkyl group; and
the carbon atom marked with “*” is racemic, substantially racemic, or enantiomerically enriched in the (S)-configuration.

Preferably, the compound of Formula (4) is the known compound Sertraline or Sertraline hydrochloride, wherein R is methyl and the compound of Formula (4) has the (1 S,4S)-configuration, which is an accessible and commercially available active pharmaceutical ingredient. Through the use of a compound of Formula (4) having a (4S)-configuration, such as Sertraline or Sertraline hydrochloride, the compound of Formula (2) is provided with the desired (S)-configuration at the 4-position (*).

Accordingly, in a first aspect of the present invention, there is provided a process for the preparation of Dasotraline (1), or a salt thereof, comprising transamination of a compound of Formula (2), with an (R)-selective ω-transaminase in the presence of an amine donor and a co-factor, wherein the carbon atom marked with “*” is racemic, substantially racemic, or enantiomerically enriched in the (S) configuration.

In a preferred embodiment of the first aspect, the process further comprises the step of purifying the product of the transamination to increase the chiral purity of the Dasotraline obtained. Preferably, the purification comprises formation and isolation of a salt formed between the Dasotraline (1) and a chiral acid. Most preferably, the chiral acid is (1R)-(−)-10-camphorsulfonic acid. Alternatively, the purification preferably comprises crystallization of Dasotraline (1) following dissolution or suspension of the product of the transamination in a solvent, optionally cooling the formed solution or suspension, and isolation of the resulting solid.

In a further preferred embodiment of the first aspect, the (R)-selective w-transaminase is selected from the group consisting of ATA-013, ATA-025, ATA-301, ATA-303, ATA-412, most preferably ATA-025, or structurally and functionally equivalent enzymes. In another preferred embodiment of the first aspect, the (R)-selective ω-transaminase is derived from an organism selected from the group consisting of Arthrobacter sp., ArRmut11, Aspergillus fumigates, Aspergillus oryzae, Aspergillus terreus, Gibberella zeae, Hyphomonas neptunium, Mycobacterium vanbaalienii, Neosartorya fischeri and Penicillium chrysogenum. Preferably, the organism is Arthrobacter sp.

In another preferred embodiment of the first aspect, the amine donor is selected from the group consisting of alanine, α-methylbenzylamine, glutamate, phenylalanine, isopropylamine, 2-aminobutane, 1,2-diaminopropane and salts thereof, most preferably the amine donor is isopropylamine.

In a further preferred embodiment of the first aspect, the transamination is conducted in an aqueous mixture comprising a solvent selected from the group consisting of acetonitrile, methanol, dimethylsulfoxide and N,N-dimethylformamide, more preferably, from dimethylsulfoxide and N,N-dimethylformamide. Most preferably, the solvent is dimethylsulfoxide. Preferably, in this embodiment, the concentration of dimethylsulfoxide in the mixture is from about 30% v/v to about 55% v/v, most preferably from about 45% v/v to about 55% v/v.

In a further preferred embodiment of the first aspect, the aqueous mixture is buffered to a pH of from about 7 to about 11.5. More preferably, the pH is from about 10 to about 11.

In a further preferred embodiment of the first aspect, the reaction is conducted at a temperature of from about 40° C. to about 65° C., and more preferably from about 50° C. to about 60° C.

In another preferred embodiment of the first aspect, the compound of Formula (2) is prepared by a process comprising oxidation of a compound of Formula (4) or a salt thereof, to provide a compound of Formula (3), and hydrolysis of the compound of Formula (3) to provide the compound of Formula (2), wherein R in the compound of Formula (4) is a C₁-C₆ alkyl group, and the carbon atom marked with “*” is racemic, substantially racemic, or enantiomerically enriched in the (S)-configuration. Preferably, in the compound of Formula (4), R is methyl. More preferably, the compound of Formula (4) has the (4S)-configuration. Most preferably, the compound of Formula (4) is Sertraline or Sertraline hydrochloride having a chiral purity in favor of the (1 S,4S)-configuration of at least about 90%, and more preferably of at least 95%.

In further preferred embodiments, the oxidation is conducted in the presence of an oxidizing agent selected from the group consisting of bromine, iodine, halosuccinimides, peroxides, hypervalent iodides, manganese oxides, oxygen/transition metal combinations, 2,3-dichloro-5,6-dicyano-1,4-benzoquinone (DDQ) and hypochlorites. Preferably, the oxidation is conducted in the presence of bromine and a metal hydroxide. Within this preferred embodiment, the hydrolysis is preferably conducted in the presence of an aqueous acid.

In another preferred embodiment of the first aspect, the carbon atom marked with “*” is enantiomerically enriched in the (S)-configuration. Preferably, the compound of Formula (2) has a chiral purity in favor of the (4S)-configuration of at least about 90%. Alternatively, the carbon atom in the compound of Formula (2) marked with “*” is racemic or substantially racemic.

In a further preferred embodiment of the first aspect, the chiral purity of Dasotraline (1) is at least about 95%.

Other aspects and features of the present invention will become apparent to those ordinarily skilled in the art upon review of the following description of specific embodiments of the invention.

### DETAILED DESCRIPTION

The present invention addresses problems associated with the known processes for the preparation of Dasotraline, wherein the inventors have unexpectedly discovered that the compound of Formula (2) is a suitable substrate for a transamination reaction mediated by an (R)-selective ω-transaminase enzyme. This approach is surprisingly capable of providing the required stereochemical configuration at the C-1 position of Dasotraline (1) regardless of the configuration at the C-4 position, allowing for the application of the transamination reaction on a racemic or substantially racemic compound of Formula (2) to afford a mixture of (cis)- and (trans)-isomers that are separable by standard purification procedures. In preferred embodiments of the present invention, wherein the process is applied to the (S)-isomer of the tetralone of Formula (2), a single step process is provided for the preparation of Dasotraline (1) having high chiral purity (greater than 95%, and preferably greater than 99%, of the desired (1 R,4S)-isomer). Alternatively, the process of the present invention can be applied to racemic tetralone of Formula (2), affording Dasotraline (1) following resolution of the transamination product.

Through the use of the processes of the present invention, it is possible to obtain Dasotraline (1) in a single step from chiral tetralone (2) while avoiding problems associated with known processes for the preparation of Dasotraline (1) such as very low yields, the use of sensitive intermediates, and the use of homogenous catalyst systems.

As used herein, the term “alkyl” means, unless otherwise stated, a straight or branched chain, saturated hydrocarbon radical having the number of carbon atoms designated (e.g., C₁-C₆ means one to six carbon atoms). When there is no indication of the number of carbon atoms in the alkyl, it is meant, unless otherwise indicated by context, that there are from 1 to 6 carbons. Preferred examples of saturated hydrocarbon groups include methyl, ethyl, n-propyl, iso-propyl, n-butyl, t-butyl, iso-butyl and sec-butyl.

As used herein, the term “ω-transaminase”, refers to an enzyme catalysing the transfer of an amino group from an amine donor to the carbonyl group of an acceptor molecule. Transaminases are classified in EC (Enzyme Commission) 2.6.1.X and ω-transaminase, in particular, corresponds with the classification code EC 2.6.1.18. The ω-transaminase may be derived from an organism, preferably a microorganism, or it may be a recombinantly produced naturally occurring (wild-type) or genetically modified transaminase. The ω-transaminase may be used in immobilised (i.e., attached to a substrate, such as a polymethacrylate or styrene/DVB copolymer resin, that is preferably porous, and that is functionalized with epoxide, amino epoxide or octadecyl groups for covalent or adsorptive attachment of the enzyme to the resin) or non-immobilised form. Substrates for immobilization are commercially available from sources such as Purolite Corporation (Bala Cynwyd, Pa.).

Preferably, the ω-transaminase is “(R)-selective”, meaning that the enzyme selectively yields an amine enriched in the (R)-configuration from the corresponding ketone. A number of “(R)-selective” ω-transaminase enzymes are described, for example, in Malik, M. S. et. al. Appl Microbiol Biotechnol. 2012, 94, 1163-71. A suitable ω-transaminase can be obtained, for example, from microorganisms like Arthrobacter sp., ArRmut11, Aspergillus fumigates, Aspergillus oryzae, Aspergillus terreus, Gibberella zeae, Hyphomonas neptunium, Mycobacterium vanbaalienii, Neosartorya fischeri and Penicillium chrysogenum. Other suitable ω-transaminase enzymes are attainable commercially from, for example, Codexis Inc. as part of the Codex® ATA Screening Kit (Product ID: ATASK-200250P) including ATA-007, ATA-013, ATA-025, ATA-117 ATA-301, ATA-303, ATA-412, ATA-P2-A01, ATA-P2-A07 and ATA-P2-B01, although structurally and functionally equivalent enzymes may also be used. Preferably, the w-transaminase is ATA-025 or a structurally and functionally equivalent enzyme. Such engineered ω-transaminases are described in, for example, U.S. Pat. No. 8,293,507 and equivalents thereof.

As used herein, the term “co-factor”, refers to a pyridoxal-5′-phosphate (PLP) or another member of the vitamin B6 family, including pyridoxine (PN), pyridoxal (PL), pyridoxamine (PM), and their phosphorylated counterparts: pyridoxine phosphate (PNP), and pyridoxamine phosphate (PMP). Preferably, the co-factor is pyridoxal-5′-phosphate (PLP).

As used herein, the term “amine donor”, refers to a compound capable of providing an amino (NH₂) group to an amine acceptor in a transamination reaction mediated by a ω-transaminase enzyme. Preferably, the amine donor is a chiral or achiral amine, including a chiral or achiral amino acid. Preferably, the amine donor is selected from the group consisting of isopropylamine (2-aminopropane), 1,2-diaminopropane, β-alanine, D,L-alanine, L-alanine, D-alanine, α-methylbenzylamine (α-MBA), glutamate, phenylalanine, glycine, 3-aminobutyrate, 2-aminobutane and γ-aminobutyrate, and salts thereof. Most preferably, the amine donor is isopropylamine (2-aminopropane).

As used herein, “room temperature” refers to a temperature of 20-25° C.

As used herein, the term “% v/v” refers to a measure of concentration and is used to express volume solute/total volume as a percentage.

As used herein, the term “about” means “close to”, and that variation from the exact value that follows the term is within amounts that a person of skill in the art would understand to be reasonable. For example, when the term “about” is used with respect to temperature, a variation of ±5° C. is generally acceptable when carrying out the processes of the present invention. When used with respect to mole equivalents, a variation of ±0.1 moles is generally acceptable. When used with respect to volumes, a variation of 10% is generally acceptable.

As used herein, the term “substantially racemic” means that there is an enantiomeric excess of less than 10%.

As used herein, the term “chiral purity” refers to the relative amount of the subject isomer with respect to the total amount of isomers of the substance, expressed as an area percentage by HPLC. For example, a compound of Formula (2) having 90% chiral purity in favor of the (4S)-isomer means that, by chiral HPLC, of the total area of peaks corresponding with the compound of Formula (2), the area of the peak corresponding with the (4S)-isomer accounts for 90%, and the area of the peak corresponding with the (4R)-isomer accounts for 10%.

In one embodiment of the present invention, Dasotraline (1) and intermediates useful in the preparation thereof are prepared by the processes set out in Schemes 3 and 4. Exemplary reagents and conditions for these reactions are disclosed herein.

In another embodiment of the present invention, there is provided a process for the preparation of the compound of Formula (3):

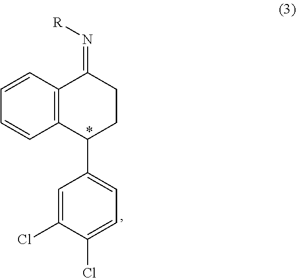

comprising oxidation of a compound of Formula (4):

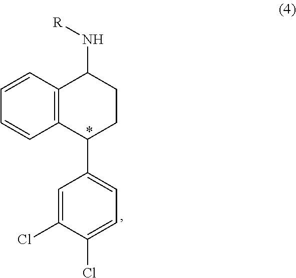

or a salt thereof,
wherein
R is a C₁-C₆ alkyl group; and
the carbon atom marked with “*” is racemic, substantially racemic, or enantiomerically enriched in the (S)-configuration.

In the compound of Formula (4), the R is preferably ethyl or methyl, and is most preferably methyl. In a further preferred embodiment, the compound of Formula (4) has the (4S)-configuration. Most preferably, the compound of Formula (4) is Sertraline or Sertraline hydrochloride.

The oxidation of the compound of Formula (4) is preferably conducted in the presence of a solvent (S1). Solvent (S1) is any suitable solvent compatible with the oxidation reaction, and is preferably selected from the group consisting of alcohols, ethers, aromatic hydrocarbons and halogenated hydrocarbons. Preferably, solvent (S1) is selected from the group consisting of methanol, ethanol, 2-propanol, tetrahydrofuran, methyl t-butyl ether, toluene and methylene chloride. Most preferably, solvent (S1) is ethanol.

The oxidation of the compound of Formula (4) is preferably conducted at a temperature of between room temperature and the boiling point of the reaction mixture, and is most preferably between about 20° C. and about 60° C.

In the oxidation of the compound of Formula (4), the oxidant may be any suitable oxidant, and is preferably selected from the group consisting bromine, iodine, halosuccinimides such as N-bromosuccinimide and N-chlorosuccinimide, peroxides such as hydrogen peroxide and t-butyl hydroperoxide (TBHP), hypervalent iodides such as 2-iodoxybenzoic (IBX) and iodosylbenzene, manganese oxides such as manganese dioxide and potassium permanganate, 2,3-dichloro-5,6-dicyano-1,4-benzoquinone (DDQ) hypochlorites such as sodium hydrochlorite, and oxygen/transition metal combinations including transition metals such as palladium, copper, ruthenium, and iron. Preferably, the oxidant is bromine used in combination with a base, preferably a metal hydroxide base such as sodium hydroxide or potassium hydroxide.

Preferably, the compound of Formula (4) is enantiomerically enriched in favour of the (S)-configuration at the 4-position (*). More preferably, the compound of Formula (4) is Sertraline, or a salt thereof, having a chiral purity in favor of the (1 S,4S)-configuration of at least about 90%, even more preferably, at least about 95%, most preferably at least about 99%. Preferably, the same configuration at the carbon atom marked with “*” is at least sustained in the compound of Formula (3).

In another embodiment of the present invention, there is provided a process for the preparation of the compound of Formula (2):

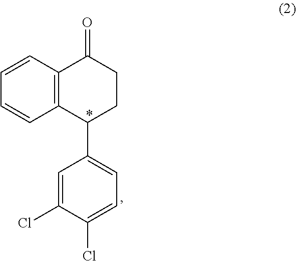 [SMILES: O=C1CCC(c2ccc(Cl)c(Cl)c2)c2ccccc21]

comprising hydrolysis of the compound of Formula (3):

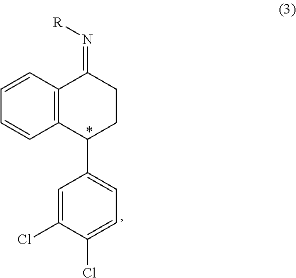

wherein
R is a C₁-C₆ alkyl group; and
the carbon atom marked with “*” is racemic, substantially racemic, or enantiomerically enriched in the (S)-configuration.

In the compound of Formula (3), the R is preferably ethyl or methyl, and is most preferably methyl. In a further preferred embodiment, the compound of Formula (3) has the (4S)-configuration.

The hydrolysis of the compound of Formula (3) is preferably conducted in the presence of an acid. The acid is any suitable inorganic or organic acid, and is preferably selected from the group consisting of hydrochloric acid, hydrobromic acid, sulfuric acid, trifluoroacetic acid, p-toluenesulfonic acid, acetic acid and citric acid. Most preferably, the acid is hydrochloric acid.

The hydrolysis of the compound of Formula (3) is conducted in the presence of water, preferably comprising a solvent (S2). Solvent (S2) is any suitable solvent compatible with the hydrolysis reaction, and is preferably selected from the group consisting of alcohols, ethers, aromatic hydrocarbons, halogenated hydrocarbons, ketones and esters. More preferably, solvent (S2) is selected from the group consisting of methanol, ethanol, 2-propanol, tetrahydrofuran, methyl t-butyl ether, toluene, methylene chloride, acetone, 2-butanone and ethyl acetate. Most preferably, solvent (S2) is methanol.

The hydrolysis of the compound of Formula (3) is preferably conducted at a temperature of between room temperature and the boiling point of the reaction mixture, and is most preferably between about 20° C. and about 60° C.

Preferably, the compound of Formula (3) is enantiomerically enriched in the (S)-configuration at the 4-position, and has a chiral purity in favor of the (4S)-configuration of at least about 90%, more preferably, at least about 95%, and most preferably, at least about 99%, with the same configuration at this carbon being at least sustained in the compound of Formula (2).

In another embodiment of the present invention, there is provided a process for the preparation of Dasotraline (1):

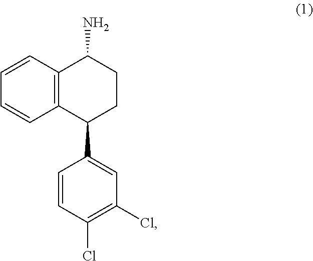 [SMILES: N[C@@H]1CC[C@@H](c2ccc(Cl)c(Cl)c2)c2ccccc21]

or a salt thereof, comprising transamination of a compound of Formula (2):

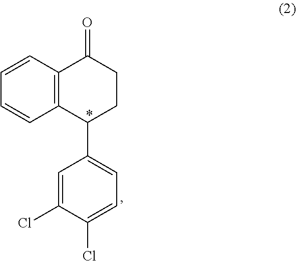 [SMILES: O=C1CCC(c2ccc(Cl)c(Cl)c2)c2ccccc21]

with an (R)-selective ω-transaminase in the presence of an amine donor and a co-factor,
wherein
the carbon atom marked with “*” is racemic, substantially racemic, or enantiomerically enriched in the (S)-configuration.

The transamination of a compound of Formula (2) is conducted using suitable reaction conditions in a reaction mixture. The reaction mixture preferably comprises the compound of Formula (2), an (R)-selective ω-transaminase, an amine donor, a co-factor, a buffer, and a solvent (S3).

In the transamination of a compound of Formula (2), the concentration or loading of the compound of Formula (2) in the reaction mixture, is at least about 0.5 g/L, and is preferably from about 0.5 g/L to about 50 g/L, more preferably from about 1 g/L to about 20 g/L, even more preferably from about 1 g/L to about 5 g/L, and most preferably from about 1 g/L to about 3 g/L.

In the transamination of a compound of Formula (2), the (R)-selective w-transaminase may be derived from an organism, preferably a microorganism, or it may be a recombinantly produced naturally occurring (wild-type) or genetically modified transaminase. The (R)-selective ω-transaminase may be used in immobilised or non-immobilised form. When immobilised, the substrate is preferably a polymethacrylate or styrene/DVB copolymer resin, that is preferably porous, and that is functionalized with epoxide, amino epoxide or octadecyl groups for covalent or adsorptive attachment of the enzyme to the resin. Preferably, the (R)-selective w-transaminase is derived from a microorganism selected from the group consisting of Arthrobacter sp., ArRmut11, Aspergillus fumigates, Aspergillus oryzae, Aspergillus terreus, Gibberella zeae, Hyphomonas neptunium, Mycobacterium vanbaalienii, Neosartorya fischeri and Penicillium chrysogenum. Preferably, the organism is Arthrobacter sp. More preferably, the (R)-selective ω-transaminase is selected from the group consisting of ATA-013, ATA-025, ATA-301, ATA-303 and ATA-412, and is most preferably ATA-025, or structurally and functionally equivalent enzymes. If desired, the enzyme may be recycled for reuse by various known techniques, such as immobilization or containment in a dialysis bag.

In the transamination of a compound of Formula (2), the amine donor is any suitable primary amine capable of transferring an amino (NH₂) group mediated by a transaminase enzyme. Preferably, the amine donor undergoes the transamination to yield a by-product that is removable following the reaction, thus off-setting the equilibrium of the transamination towards Dasotraline (1). For example, the by-product may be volatile, such as the acetone produced when isopropylamine is used as the amine donor, allowing for removal of the by-product via the passage of an inert gas into the reaction medium, or by evaporative methods. Alternatively, the by-product may be a substrate for a further enzymatic reaction, such as the pyruvate produced when alanine is used as the amine donor, allowing for removal of the by-product by treatment with a pyruvate decarboxylase.

Preferably, the amine donor is a chiral or achiral amine, including a chiral or achiral amino acid. Preferably, the amine donor is selected from the group consisting of isopropylamine, 1,2-diaminopropane, β-alanine, D,L-alanine, L-alanine, D-alanine, α-methylbenzylamine, glutamate, phenylalanine, glycine, 3-aminobutyrate, 2-aminobutane, γ-aminobutyrate and salts thereof. Most preferably, the amine donor is isopropylamine or a salt thereof. The amine donor is preferably used in molar excess, preferably at least about 100 mole equivalents with respect to the compound of Formula (2). The concentration or loading of the amine donor in the reaction mixture is preferably at least about 0.5 M, and is more preferably from about 0.5 M to about 2 M, even more preferably from about 0.5 M to about 1.5 M, and most preferably about 0.5 M to about 1.0 M.

In the transamination of a compound of Formula (2), the co-factor is preferably pyridoxal-5′-phosphate (PLP). Alternatively, the co-factor is another member of the vitamin B6 family selected from the group consisting of pyridoxine (PN), pyridoxal (PL), pyridoxamine (PM), and their phosphorylated counterparts: pyridoxine phosphate (PNP), and pyridoxamine phosphate (PMP). Preferably, the co-factor is used in amount sufficient to promote the reaction, with the concentration of co-factor in the reaction mixture preferably being at least about 0.1 mole equivalents with respect to the compound of Formula (2). The concentration or loading of the amine donor in the reaction mixture is preferably at least about 0.5 mM, more preferably from about 0.5 mM to about 2 mM, even more preferably from about 0.5 mM to about 1.5 mM, and most preferably from about 0.5 mM to about 1.0 mM.

The transamination of a compound of Formula (2) is preferably conducted in the presence of an aqueous buffer system. The buffer is chosen to maintain the pH in the desired range, which is preferably from about pH 7 to about pH 11.5, more preferably from about pH 9 to about pH 11, and most preferably from about pH 10 to about 11. Preferably, the buffer is selected from the group consisting of triethanolamine, potassium phosphate and sodium borate. The concentration of the buffer in the reaction mixture is preferably from about 50 mM to about 150 mM, and is more preferably from about 50 mM to about 100 mM.

The transamination of a compound of Formula (2) is preferably conducted in the presence of a suitable solvent (S3). Preferably, the solvent (S3) is selected from the group consisting of acetonitrile, methanol, N,N-dimethylformamide and dimethylsulfoxide. More preferably, solvent (S3) is N,N-dimethylformamide and dimethylsulfoxide, and is most preferably dimethylsulfoxide. The concentration of the solvent (S3) in the reaction mixture is preferably at least about 30% v/v, more preferably from about 30% v/v to about 60% v/v, even more preferably from about 30% v/v to about 55% v/v, and most preferably from about 45% v/v to about 55% v/v.

The transamination of a compound of Formula (2) is conducted at a temperature suitable for the transaminase, and is preferably conducted at a temperature from about 40° C. to about 65° C., more preferably from about 50° C. to about 60° C.

In a preferred embodiment of the present invention, the transamination is conducted using the compound of Formula (2) has a chiral purity in favor of the (4S)-configuration of at least about 90%, more preferably, at least about 95%, and most preferably, at least about 99%, with the configuration at this carbon being at least sustained in the resulting Dasotraline (1). In this preferred embodiment, transamination of the compound of Formula (2) provides the compound of Formula (1) in favour of the (1 R,4S)-trans-configuration.

Alternatively, in another embodiment of the present invention, the transamination is conducted using the compound of Formula (2) that is racemic or substantially racemic. In this embodiment, transamination of the compound of Formula (2) having equimolar or approximately equimolar amounts of the (R)- and (S)-enantiomers provides Dasotraline (1) as a mixture in favor of the (1 R,4S)-trans-configuration and the (1 R,4R)-cis-configuration, with lesser amounts of the (1 S,4S)-cis- and (1 S,4R)-trans-configurations (i.e., the configuration of C-1 is selectively (R)).

The chiral purity of Dasotraline (1), or a salt thereof, whether produced from racemic, substantially racemic, or enantiomerically enriched compound of Formula (2), can be further enhanced by purification, preferably by direct crystallization or resolution using a chiral acid. Direct crystallization comprises dissolving or suspending Dasotraline (1), or a salt thereof, in a suitable solvent (S4) at a suitable temperature, preferably between about 20° C. to about 70° C., followed by cooling, if necessary, and isolation of the resulting solid. Resolution using a chiral acid comprises forming an acid salt of Dasotraline (1) with a chiral acid in a suitable solvent (S4), as described above, at a suitable temperature, preferably between about 20° C. to about 70° C., followed by cooling, if necessary, and isolation of the resulting solid. Alternatively, in this crystallization process, purified Dasotraline (1), or a salt thereof, may be recovered from the mother liquor, whereas the impurities are enriched in the crystallized solid.

The suitable solvent (S4) is preferably selected from the group consisting of nitriles, alcohols, esters, aromatic hydrocarbons and ethers; more preferably, solvent (S4) is selected from the group consisting of acetonitrile, methanol, 2-propanol, ethyl acetate, toluene, methyl t-butyl ether, and tetrahydrofuran. Most preferably, solvent (S4) is toluene.

Resolution using a chiral acid comprises forming an acid salt of Dasotraline (1) with a chiral acid in a suitable solvent (S4), as described above, at a suitable temperature, preferably between about 20° C. to about 70° C., followed by cooling, if necessary, and isolation of the resulting solid. Alternatively, in this resolution process, the acid salt of Dasotraline (1) with a chiral acid may be recovered from the mother liquor, whereas the impurities are enriched in the crystallized solid. Preferably, the chiral acid is selected from the group consisting of the optical isomers of di-p-toluoyl tartaric acid, mandelic acid, 10-camphorsulfonic acid, tartaric acid, dibenzoyl tartaric acid and malic acid. More preferably, the chiral acid is selected from the group consisting of (D)-(−)-tartaric acid, (R)-(−)-mandelic acid, (1R)-(−)-10-camphorsulfonic acid, and (+)-O,O′-di-p-toluoyl-D-tartaric acid. Most preferably, the chiral acid is (1R)-(−)-10-camphorsulfonic acid, and the solvent (S4) is ethyl acetate. Following isolation of the salt, Dasotraline (1) is obtained using standard methods, such as dissolution in an aqueous medium, treatment with a base such as sodium hydroxide, sodium bicarbonate or ammonia, extraction into an organic solvent, and removal of the solvent. Alternatively, Dasotraline (1) can be obtained as a salt, for example, the hydrochloride salt, by addition of a suitable acid to the organic solvent.

Purification of Dasotraline (1), or a salt thereof, can be conducted by either or both of these methods. Preferably, following purification, the chiral purity of Dasotraline (1), or a salt thereof, is at least 90%, more preferably at least 95%, and most preferably at least 99%.

In a preferred method, the chiral purity of Dasotraline (1) is enhanced by combining Dasotraline (1) with (1R)-(−)-10-camphorsulfonic acid (1 equivalent based on the estimated amount of the (1 R,4S)-isomer of Dasotraline (1) present) and ethyl acetate (approximately 4-5 mL per gram Dasotraline (1)) to afford a white suspension, which is heated to about 40° C. for around 1 hour, before being slowly cooled to room temperature over approximately 2 hours, and maintained at this temperature for about 18 hours. The resulting solid can be collected by filtration and washed with ethyl acetate, and dried in vacuo to provide Dasotraline (1R)-(−)-10-camphorsulfonate as a white solid. Dasotraline (1), or a pharmaceutically acceptable salt thereof, is then obtained from the Dasotraline (1R)-(−)-10-camphorsulfonate by standard methods, as discussed above.

### EXAMPLES

The following examples are illustrative of some of the embodiments of the invention described herein. It will be apparent to the skilled reader that various alterations to the described processes in respect of the reactants, reagents and conditions may be made when using the processes of the present invention without departing from the scope or intent thereof.

### Analysis Method for Determining the Chromatographic Purity of Dasotraline (1)

The method shown in Table 1 was used to determine the chromatographic purity of samples of Dasotraline (1) as provided in the examples that follow.

TABLE 1
HPLC method for the determination of chromatographic purity of
Dasotraline (1).

| Instrument | Waters 2695 Separations Module HPLC | | |
|---|---|---|---|
| Column | ZORBAX SB-CN, 4.6 × 150 mm, 3.5 μm | | |
| Column | 35° C. | | |
| Temp. | | | |
| Sample | 20-25° C. | | |
| temp. | | | |
| Mobile | Solution A: Sodium dodecylsulfate (1.44 g) in | | |
| phase | 1000 mL nanopure water (avoid shaking), | | |
| | followed by addition of 2 mL | | |
| | 85% phosphoric acid; | | |
| | Mobile Phase A: 800 mL solution A and 200 | | |
| | mL HPLC grade acetonitrile; | | |
| | Mobile Phase B: HPLC grade acetonitrile | | |
| | Time | % Solution A | % Solution B |
| Gradient | 0.0 | 85 | 15 |
| Program | 20.00 | 40 | 60 |
| | 20.10 | 5 | 95 |
| | 23.00 | 85 | 15 |

| | |
|---|---|
| Flow rate | 1.0 mL/minute |
| Injection | 10 μL |
| volume | |
| Detector | 220 nm |
| Run time | 28 minutes |
| Sample prep. | Mix 1 mL of the supernatant of the |
| | reaction mixture with 1 mL of |
| | acetonitrile and filter through a 0.45 μm filter |

### Analysis Method for Determination of the Chiral Purity of Dasotraline (1)

The method shown in Table 2 was used to determine the chiral purity of samples of Dasotraline (1) and salts thereof as provided in the examples that follow.

TABLE 2
HPLC method for the determination of chiral purity of the
compound of Formula (1)

| | |
|---|---|
| Instrument | Agilent 1100 series HPLC |
| Column | Chiralpak ® AD-H, 4.6 × 250 mm, 5 μm, |
| | DAICEL |
| Column Temp. | 25° C. |
| Sample temp. | 20-25° C. |
| Mobile phase | 950 mL of hexane, 50 mL of ethanol and 0.7 mL |
| | of ethanesulfonic Acid (70% aqueous solution). |
| Mode | Isocratic |
| Flow rate | 1.0 mL/minute |
| Injection volume | 10 μL |
| Detector | 220 nm |
| Run time | 40 minutes |
| Sample prep. | 1-2 mg of sample is dissolved in 1 mL ethanol |
| | followed by addition of 2 mL mobile phase. |

### Preparation of Buffer Solutions

Buffer solutions containing the amine donor and co-factor for use in Examples 3, 4 and 5 were prepared as follows:

1. Buffer A: Isopropylamine (2.43 g, 41.1 mmol), boric acid (245 mg, 4.0 mmol) and pyridoxal 5′-phosphate (PLP) (8.3 mg, 0.03 mmol) were combined in water (10 mL) with stirring until complete dissolution. The pH of the solution was adjusted to 10.5 using concentrated aqueous hydrogen chloride, and the final volume was adjusted to 30 mL with water.
2. Buffer B: Isopropylamine (3.65 g, 61.8 mmol), boric acid (368 mg, 6.0 mmol) and pyridoxal 5′-phosphate (PLP) (12 mg, 0.05 mmol) were combined in water (10 mL) with stirring until complete dissolution. The pH of the solution was adjusted to 10.5 using concentrated aqueous hydrogen chloride, and the final volume was adjusted to 40 mL with water.
3. Buffer C: The solution is prepared in accordance with Buffer A, however the pH is adjusted to 11.5 rather than 10.5.
4. Buffer D: The solution is prepared in accordance with Buffer A, however the pH is adjusted to 9.5 rather than 10.5.
5. Buffer E: 1,2-Diaminopropane (3.02 g, 40.7 mmol), boric acid (245 mg, 4.0 mmol) and pyridoxal 5′-phosphate (PLP) (8 mg, 0.03 mmol) were combined in water (10 mL) with stirring until complete dissolution. The pH of the solution was adjusted to 9.9 using concentrated aqueous hydrogen chloride, and the final volume was adjusted to 30 mL with water.
6. Buffer F: Isopropylamine (2.43 g, 41.1 mmol), boric acid (245 mg, 4.0 mmol) and pyridoxal 5′-phosphate (PLP) (8.3 mg, 0.03 mmol) were combined in water (10 mL) with stirring until complete dissolution. The pH of the solution was adjusted to 7.5 using concentrated aqueous hydrogen chloride, and the final volume was adjusted to 30 mL with water.

### Example 1: Preparation of N-[(4S)-4-(3,4-dichlorophenyl)-3,4-dihydro-1(2H)-naphthalenylidene]methanamine (Compound of Formula (3-A))

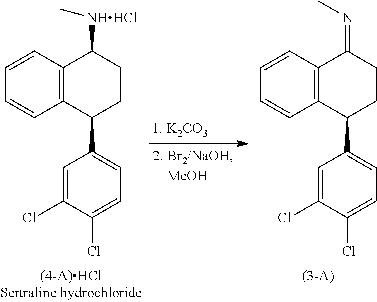

Sertraline hydrochloride (11.2 g, 32.7 mmol) was partitioned between dichloromethane (100 mL) and a saturated potassium carbonate aqueous solution (100 mL). The organic layer was separated, concentrated to dryness, and the oily residue was dissolved in methanol (90 mL). Sodium hydroxide (7.9 g, 198 mmol) was added to the solution, followed by bromine (6.0 g, 37.5 mmol), while controlling the temperature to under 35° C. throughout the latter addition. After stirring for 2 hours, the solid formed was collected by filtration and washed with methanol (2×10 mL). The product was then suspended in water (50 mL), stirred vigorously for 20 minutes, and collected by filtration. The isolated solids were washed with water (3×25 mL) and allowed to air-dry to afford the compound of Formula (3-A) as a white solid (9.7 g, 98% yield).

### Example 2: Preparation of (4S)-4-(3,4-dichlorophenyl)-3,4-dihydro-1(2H)-naphthalenone (Compound of Formula (2-A))

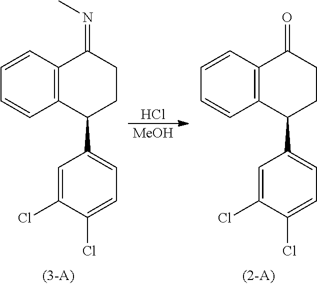 [SMILES: "MeOH" ; C/N=C1/CC[C@@H](c2ccc(Cl)c(Cl)c2)c2ccccc21 ; Cl ; O=C1CC[C@@H](c2ccc(Cl)c(Cl)c2)c2ccccc21]

The compound of Formula (3-A) (6.1 g, 20.1 mmol) was dissolved in a mixture of methanol (40 mL) and concentrated aqueous hydrochloric acid (8 mL). After stirring for 48 hours at ambient temperature, the resulting solid was collected by filtration, washed with water (3×20 mL), and dried in vacuo to afford the compound of Formula (2-A) (4.4 g, 75% yield).

### Example 3: Preparation of (1 R,4S)-4-(3,4-dichlorophenyl)-1,2,3,4-tetrahydronaphthalen-1-amine (Dasotraline (1) hydrochloride) Using ATA-025

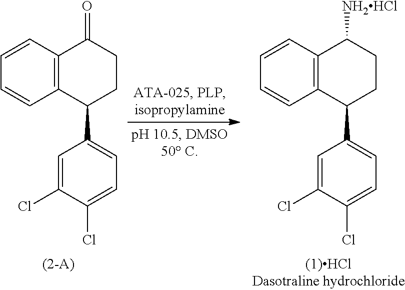

An aliquot of Buffer A (18 mL) was slowly added to a solution of the compound of Formula (2) (62 mg, 0.21 mmol) in dimethylsulfoxide (18 mL) at room temperature, followed by the addition of enzyme ATA-025 (201 mg). The resulting milky solution was heated to 50° C. with stirring, and the progress of the reaction was followed by HPLC. After 22 hours, the conversion to Dasotraline (1) was approximately 75%. A second portion of enzyme ATA-025 (77.5 mg) was added, and the reaction continued for 24 hours, following which the conversion to Dasotraline (1) had reached 84%. Following addition of a third portion of enzyme ATA-025 (85 mg), no further progress of the reaction was observed. The reaction was cooled to room temperature and isopropyl acetate (35 mL) was added with vigorous stirring followed by separation of the organic phase by centrifugation. The aqueous phase was treated with isopropyl acetate (35 mL), and the centrifugation process was repeated. The combined organic layers were washed with water (5×35 mL) to remove residual dimethylsulfoxide from the organic phase, which was separated and dried over anhydrous sodium sulfate, filtered, and concentrated to dryness to afford a crude oily residue (60.4 mg). The residue was dissolved in dichloromethane (1 mL) and treated with hydrogen chloride solution (4M in dioxane, 0.1 mL). The resulting solid was filtered to afford Dasotraline (1) hydrochloride (28.4 mg, 45% yield). Chromatographic purity (HPLC, area %): 98.7%; chiral purity (chiral HPLC, area %) in favor of the (1 R,4S)-isomer: 100%.

### Example 4: Preparation of Dasotraline (1)

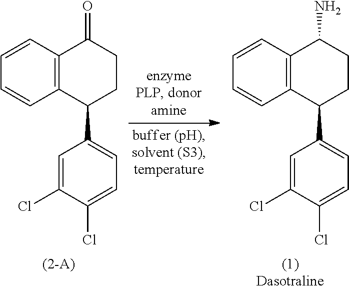 [SMILES: O=C1CC[C@@H](c2ccc(Cl)c(Cl)c2)c2ccccc21 ; N[C@@H]1CC[C@@H](c2ccc(Cl)c(Cl)c2)c2ccccc21]

Embodiments of Example 4 under various conditions

### Example 4A: Enzyme=ATA-025, Amine Donor=Isopropylamine, Buffer=Buffer B (pH 10.5), Solvent (S3)=dimethylsulfoxide, Temperature=50° C.

Buffer B (40 mL) was slowly added to a solution of the compound of Formula (2-A) (140 mg, 0.48 mmol) in dimethylsulfoxide (40 mL) at room temperature, followed by the addition of enzyme ATA-025 (401 mg). The resulting milky solution was heated to 50° C. with stirring, and the progress of the reaction was followed by HPLC. After 5 days, the conversion to Dasotraline (1) was approximately 83%. The reaction mixture was cooled to room temperature and isopropyl acetate (80 mL) was added with vigorous stirring followed by separation of the organic phase by centrifugation. The aqueous phase was treated with isopropyl acetate (80 mL), and the centrifugation process repeated. The combined organic layers were washed with water (5×80 mL) to remove residual dimethylsulfoxide from the organic phase, which was separated, dried over anhydrous sodium sulfate, filtered and concentrated to dryness to afford a crude oily residue (149 mg). The residue was purified by preparative column chromatography (gradient elution 2-10% methanol containing 1% ammonium hydroxide in dichloromethane, Innoflash® 12 g column) to afford Dasotraline (1) (81.2 mg, 58% yield) as an oil. Chromatographic purity (HPLC, area %): 99.2%; chiral purity (chiral HPLC, area %) in favor of the (1 R,4S)-isomer: 99.6% (99.2% ee).

### Example 4B: Enzyme=ATA-025, Amine Donor=Isopropylamine, Buffer=Buffer C (pH 11.5), Solvent (S3)=dimethylsulfoxide, Temperature=50° C.

An aliquot of Buffer C (900 μL) was slowly added to a solution of the compound of Formula (2-A) (3.1 mg, 0.01 mmol) in dimethylsulfoxide (900 μL) at room temperature, followed by the addition of enzyme ATA-025 (10 mg). The resulting milky solution was heated to 50° C. with stirring, and the progress of the reaction was followed by HPLC. After 1 week, the conversion reached to Dasotraline (1) was approximately 84%.

### Example 4C: Enzyme=ATA-025, Amine Donor=Isopropylamine, Buffer=Buffer D (pH 9.5), Solvent (S3)=dimethylsulfoxide, Temperature=50° C.

An aliquot of Buffer D (900 μL) was slowly added to a solution of the compound of Formula (2-A) (3.1 mg, 0.01 mmol) in dimethylsulfoxide (300 μL) at room temperature, followed by the addition of enzyme ATA-025 (11 mg). The resulting milky solution was heated to 50° C. with stirring, and the progress of the reaction was followed by HPLC. After 52 hours, the conversion to Dasotraline (1) was approximately 73%.

### Example 4D: Enzyme=ATA-025, Amine Donor=Isopropylamine, Buffer=Buffer A (pH 10.5), Solvent (S3)=N,N-dimethylformamide, Temperature=50° C.

An aliquot of Buffer A (900 μL) was slowly added to a solution of the compound of Formula (2-A) (3.1 mg, 0.01 mmol) in N,N-dimethylformamide (900 μL) at room temperature, followed by the addition of enzyme ATA-025 (10 mg). The resulting milky solution was heated to 50° C. with stirring, and the progress of the reaction was followed by HPLC. After 71 hours, the conversion to Dasotraline (1) reached 84%.

### Example 4E: Enzyme=ATA-025, Amine Donor=Isopropylamine, Buffer=Buffer A (pH 10.5), Solvent (S3)=dimethylsulfoxide, Temperature=60° C.

An aliquot of Buffer A (990 μL) was slowly added to a solution of the compound of Formula (2-A) (3.4 mg, 0.01 mmol) in dimethylsulfoxide (990 μL) at room temperature, followed by the addition of enzyme ATA-025 (11 mg). The resulting milky solution was heated to 60° C. with stirring, and the progress of the reaction was followed by HPLC. After 122 hours, the conversion to Dasotraline (1) reached 83%.

### Example 4F: Enzyme=ATA-025, Amine Donor=1,2-diaminopropane, Buffer=Buffer E (pH 9.9), Solvent (S3)=dimethylsulfoxide, Temperature=50° C.

An aliquot of Buffer E (900 μL) was slowly added to a solution of the compound of Formula (2-A) (3.1 mg, 0.01 mmol) in dimethylsulfoxide (900 μL) at room temperature, followed by the addition of enzyme ATA-025 (10 mg). The resulting milky solution was heated to 50° C. with stirring, and the progress of the reaction was followed by HPLC. After 99 hours, the conversion to Dasotraline (1) reached 84%.

### Example 4G: Enzyme=ATA-303, Amine Donor=Isopropylamine, Buffer=Buffer D (pH 9.5), Solvent (S3)=dimethylsulfoxide, Temperature=50° C.

An aliquot of Buffer D (900 μL) was slowly added to a solution of the compound of Formula (2-A) (3.1 mg, 0.01 mmol) in dimethylsulfoxide (900 μL) at room temperature, followed by the addition of enzyme ATA-303 (11 mg). The resulting milky solution was heated to 50° C. with stirring, and the progress of the reaction was followed by HPLC. After 1 week, the conversion to Dasotraline (1) reached 64%.

### Example 4H: Enzyme=ATA-415, Amine Donor=Isopropylamine, Buffer=Buffer D (pH 9.5), Solvent (S3)=dimethylsulfoxide, Temperature=50° C.

An aliquot of Buffer D (900 μL) was slowly added to a solution of the compound of Formula (2-A) (3.1 mg, 0.01 mmol) in dimethylsulfoxide (900 μL) at room temperature, followed by the addition of enzyme ATA-415 (10 mg). The resulting milky solution was heated to 50° C. with stirring, and the progress of the reaction was followed by HPLC. After 1 week, the conversion to Dasotraline (1) reached 42%.

### Example 41: Enzyme=ATA-013, Amine Donor=Isopropylamine, Buffer=Buffer F (pH 7.5), Solvent (S3)=dimethylsulfoxide, Temperature=50° C.

An aliquot of Buffer F (900 μL) was slowly added to a solution of the compound of Formula (2-A) (3.1 mg, 0.01 mmol) in dimethylsulfoxide (900 μL) at room temperature, followed by the addition of enzyme ATA-013 (11 mg). The resulting milky solution was heated to 50° C. with stirring, and the progress of the reaction was followed by HPLC. After 2 days, the conversion to Dasotraline (1) reached 29%.

### Example 4J: Enzyme=Recycled ATA-025, Amine Donor=Isopropylamine, Buffer=Buffer D (pH 9.5), Solvent=dimethylsulfoxide, Temperature=50° C.

A further reaction conducted under the conditions of Example 4C was subjected to the aqueous work-up described in Example 4A. A sample of the aqueous phase obtained from the work-up, which contained recovered ATA-025 (approximately 112 mg), was treated with the compound of Formula (2-A) (23 mg, 0.08 mmol). The resulting suspension was heated to 50° C. with stirring, and the progress of the reaction was followed by HPLC. After approximately 1 week, the reaction reached a maximum conversion to Dasotraline (1) of approximately 62%, which was unchanged following addition of further enzyme.

### Example 5: Preparation of (1R,4S)/(1R,4R)-4-(3,4-dichlorophenyl)-1,2,3,4-tetrahydronaphthalen-1-amine (Compound of Formula (1-B)) using ATA-025

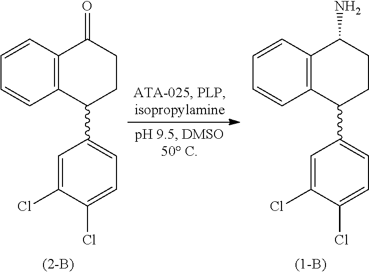 [SMILES: O=C1CCC(c2ccc(Cl)c(Cl)c2)c2ccccc21 ; N[C@@H]1CCC(c2ccc(Cl)c(Cl)c2)c2ccccc21]

An aliquot of Buffer D (900 μL) was slowly added to a solution of the compound of Formula (2-B) (3.1 mg, 0.01 mmol) in dimethylsulfoxide (900 μL) at room temperature, followed by the addition of enzyme ATA-025 (11 mg). The resulting milky solution was heated to 50° C. with stirring, and the progress of the reaction was followed by HPLC. After 49 hours, the conversion to the compound of Formula (1-B) reached 57%.

What is claimed is:

1. A process for the preparation of Dasotraline (1): 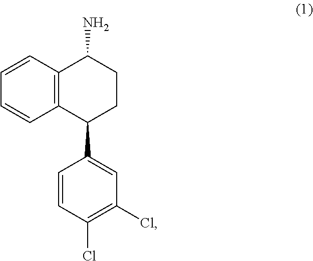 [SMILES: N[C@@H]1CC[C@@H](c2ccc(Cl)c(Cl)c2)c2ccccc21]
or a salt thereof, comprising transamination of a compound of Formula (2): 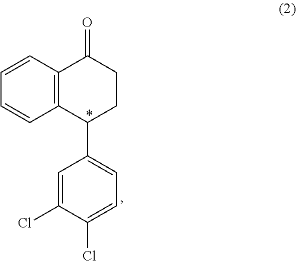 [SMILES: O=C1CCC(c2ccc(Cl)c(Cl)c2)c2ccccc21]
with an (R)-selective ω-transaminase in the presence of an amine donor and a co-factor,
wherein
the carbon atom marked with “*” is racemic, substantially racemic, or enantiomerically enriched in the (S)-configuration.

2. The process of claim 1 further comprising the step of purifying the product of the transamination to increase the chiral purity of the Dasotraline (1) obtained.

3. The process of claim 2, wherein the purification comprises formation and isolation of a salt formed between Dasotraline (1) and a chiral acid.

4. The process of claim 3, wherein the chiral acid is (1R)-(−)-10-camphorsulfonic acid.

5. The process of claim 1, wherein the (R)-selective ω-transaminase is selected from the group consisting of ATA-013, ATA-025, ATA-301, ATA-303, ATA-412, or structurally and functionally equivalent enzymes.

6. The process of claim 5, wherein the (R)-selective ω-transaminase is ATA-025 or a structurally and functionally equivalent enzyme.

7. The process of claim 1, wherein the (R)-selective ω-transaminase is derived from an organism selected from the group consisting of Arthrobacter sp., ArRmut11, Aspergillus fumigates, Aspergillus oryzae, Aspergillus terreus, Gibberella zeae, Hyphomonas neptunium, Mycobacterium vanbaalienii, Neosartorya fischeri and Penicillium chrysogenum.

8. The process of claim 5, wherein the amine donor is selected from the group consisting of alanine, α-methylbenzylamine, glutamate, phenylalanine, isopropylamine, 2-aminobutane, 1,2-diaminopropane and salts thereof.

9. The process of claim 8, wherein the amine donor is isopropylamine.

10. The process of claim 8, wherein the transamination is conducted in an aqueous mixture comprising a solvent selected from the group consisting of dimethylsulfoxide and N,N-dimethylformamide.

11. The process of claim 10, wherein the solvent is dimethylsulfoxide.

12. The process of claim 11, wherein the concentration of dimethylsulfoxide in the mixture is from about 30% v/v to about 55% v/v.

13. The process of claim 11, wherein the aqueous mixture is buffered to a pH of from about 7 to about 11.5.

14. The process of claim 13, wherein the reaction is conducted at a temperature of from about 40° C. to about 65° C.

15. The process of claim 1, wherein the compound of Formula (2) is prepared by a process comprising:
(i) oxidation of a compound of Formula (4): 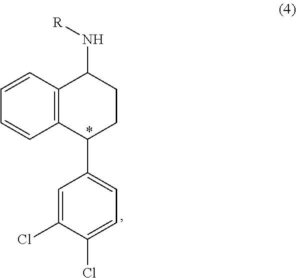
or a salt thereof, to provide a compound of Formula (3): 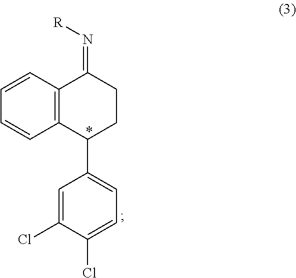
and
(ii) hydrolysis of the compound of Formula (3) to provide the compound of Formula (2),
wherein
R is a C₁-C₆ alkyl group; and
the carbon atom marked with “*” is racemic, substantially racemic, or enantiomerically enriched in the (S)-configuration.

16. The process of claim 15, wherein R is methyl.

17. The process of claim 15, wherein the oxidation is conducted in the presence of an oxidizing agent selected from the group consisting of bromine, iodine, halosuccinimides, peroxides, hypervalent iodides, manganese oxides, oxygen/transition metal combinations, 2,3-dichloro-5,6-dicyano-1,4-benzoquinone (DDQ) and hypochlorites.

18. The process of claim 15, wherein the oxidation is conducted in the presence of bromine and a metal hydroxide.

19. The process of claim 15, wherein the hydrolysis is conducted in the presence of an aqueous acid.

20. The process of claim 5, wherein the carbon atom marked with “*” is enantiomerically enriched in the (S)-configuration and the compound of Formula (2) has a chiral purity in favor of the (4S)-configuration of at least about 90%.
